(19) United States
(12) Patent Application Publication
(10) Pub. No.: US 2011/0189220 A1
(43) Pub. Date: Aug. 4, 2011

(54) MUSHROOM COMPOSITIONS AND METHODS FOR MAKING AND USING

(75) Inventors: Peiying YANG, Sugar Land, TX (US); Robert NEWMAN, Surry, ME (US); Paul SCHULICK, Brattleboro, VT (US)

(73) Assignees: NEW CHAPTER INC., Brattleboro, VT (US); BOARD OF REGENTS OF THE UNIVERSITY OF TEXAS SYSTEM, Austin, TX (US)

(21) Appl. No.: 13/016,498

(22) Filed: Jan. 28, 2011

Related U.S. Application Data
(60) Provisional application No. 61/282,376, filed on Jan. 29, 2010.

(51) Int. Cl. A61K 36/06; A61P 3/10; A61P 19/02; A61P 1/16; A61P 9/10; A61P 29/00; A61P 11/00; A61P 17/06; A61P 25/16

(52) U.S. Cl. 424/195.15

(57) ABSTRACT

The present subject matter relates to a novel mushroom composition and methods for making and using the same. In one aspect, the subject matter involves a composition comprising a combination of mushrooms or components derived from mushrooms selected from the group consisting of Reishi Ganoderma lucidum, Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Coriolus Tramentes versicolor, Chaga Inonotus obliquus, and Chaga Inonotus obliquus. In some embodiments, the present subject matter relates to methods for modulating immune function, regulating the activity of lipoxygenases and cyclooxygenases, improving cardiovascular health, and/or inhibiting cell proliferation diseases and disorders. In one embodiment the composition provides a balancing of anti-inflammatory and pro-inflammatory function in an animal, including in humans.

### CROSS REFERENCE TO RELATED APPLICATIONS

This application claims the benefit of U.S. Provisional Patent Application No. 61/282,376, filed Jan. 29, 2010, which is incorporated herein by reference in its entirety.

### BACKGROUND

The immune system is the body's basic protection and serves as both its essential mechanism for healing and its defense against wounds, foreign bodies such as bacteria, viruses, splinters and anything recognized as ‘non-self’. In addition, the immune system also provides a mechanism of learning about and defending against foreign antigens in order to enable development of antibodies and cellular based clearance of foreign matter. The immune system is also linked to cellular production of acute pro- and anti-inflammatory mediators which play a significant role in defense against wounds and induction of tissue repair. In general, it is believed that foodstuffs such as mushrooms, garlic, ginseng, turmeric, and the like are effective in the promotion of human health which is achieved and maintained in part through maintenance of active and effective immune function in the body. It is well known there are many vitamins and minerals, essential fatty acids, proteins, and carbohydrates which contribute to healthy immune function. Inadequate consumption of vitamins, minerals, and essential-unsaturated fatty acids have been linked to various diseases and disorders, particularly age-related diseases and disorders such as arthritis, cancer, cardiac dysfunction, as well as others.

Various mushroom species have been employed in traditional herbal medicine, and their role in supporting immune function is currently being investigated in the scientific community. For example, all plants produce certain kinds of sugars that are a source of energy and that form the cell walls in some plants. Some of the complex sugars in mushrooms are called alpha and beta glucans and are the focus of studies concerning their effects on the human immune system. Beta glucans are generally not produced naturally in humans, and must therefore come from plant and animal sources. Maitake mushrooms, for example, are exceptionally high in beta glucans, while shiitake mushrooms have high concentrations of alpha glucans.

Nutriceuticals and dietary supplements are becoming increasingly popular as research uncovers specific compounds or compositions contained in food that have therapeutic effects, including immunomodulatory properties. There is a continuing need for nutriceuticals and dietary supplements which provide new formulations that enhance immune system function in new and unexpected ways.

### SUMMARY

The present subject matter relates to a novel mushroom composition and methods for making and using the same. In one aspect, the subject matter involves a composition comprising a combination of mushrooms or components derived from mushrooms selected from the group consisting of Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Coriolus Tramentes versicolor, and Chaga Inonotus obliquus. In some embodiments, the present subject matter relates to methods for modulating immune function, regulating inflammatory response through activity of lipoxygenases and cyclooxygenases, improving cardiovascular health, and/or inhibiting cell proliferative diseases and disorders. In one embodiment the composition provides a beneficial balancing of anti-inflammatory and pro-inflammatory function in animals, including in humans. In another embodiment the composition provides for antioxidant protection within human cells.

### BRIEF DESCRIPTION OF THE DRAWINGS

FIG. 1 shows the effect of a mushroom formulation of the present subject matter on the formation of COX-2 derived metabolites in rat macrophage Raw264.7 cells. Production of pro-inflammatory lipid mediators PGE₂ (FIG. 1A) and PGF2-alpha (FIG. 1B) was decreased by administering the mushroom formulation.

FIG. 2 shows the effect of a mushroom formulation of the present subject matter on the formation of COX-2 derived metabolites in rat macrophage Raw264.7 cells. Production of anti-inflammatory lipid mediators 15-keto-PGE₂ (FIG. 2A), PGD₂ (FIG. 2B), and 13-PGD₂ (FIG. 2C) was increased by administering the mushroom formulation.

FIG. 3 shows the effect of a mushroom formulation of the present subject matter on the formation of Lipoxygenase derived metabolites in rat macrophage Raw264.7 cells. Production of pro-inflammatory 12-LOX product 12-HETE (FIG. 3A) was decreased. Production of anti-inflammatory 15-LOX-1 product 13-HODE (FIG. 3B) was increased.

FIG. 4 shows the effect of a mushroom formulation of the present subject matter on the formation of Lipoxygenase derived metabolites in rat macrophage Raw264.7 cells. Other 5-LOX derived metabolites LTB₄ (FIG. 4A) and the 15-LOX metabolite, 15-HETE, (FIG. 4B) exhibited minimal changes after administering the mushroom formulation.

FIG. 5 shows the expression of inflammatory associated genes in Raw cells and the effect of administering a mushroom formulation of the present subject matter. Expression of the following genes are provided: (1) adrenergic receptor, beta 1; (2) adrenergic receptor, beta 2; (3) annexin A3; (4) calcium channel, voltage-dependent beta 4 subunit; (5) cysteinyl leukotriene receptor 1; (6) hydroxyprostaglandin dehydrogenase 15 (NAD) also known as 15-PGDH or 15-prostaglandin dehydrogenase; (7) histamine receptor H1; (8) integrin alpha L; (9) leukotriene A4 hydrolase. The data demonstrates that the formulation is capable of increased expression of enzymes such as 15-PGDH, which is a well established tumor suppressor gene.

FIG. 6 shows the expression of inflammatory associated genes in Raw cells and the effect of administering a mushroom formulation of the present subject matter. Expression of the following genes are provided: (1) arachidonate 5-lipoxygenase; (2) histamine receptor H2; (3) interleukin 1 receptor, type I; (4) phospholipase A2, group X; (5) phospholipase A2, group IB; (6) phospholipase C, delta 1; (7) prostaglandin F receptor; (8) tumor necrosis factor. The data demonstrates that the formulation is capable of decreased expression of inflammatory associated genes.

FIG. 7 shows the production of TNF-α in Raw cells (0.5×10⁶/ml) treated with a mushroom formulation of the present subject matter. The data demonstrates that the formulation stimulates the production of TNF-α, an important component of acute inflammatory defense reactions.

FIG. 8 shows the production of TNF-α in Raw cells ((0.5×10⁶/ml) treated with various mushroom formulations of the present subject matter. The data demonstrates that the formulation stimulates the production of TNF-α.

FIG. 9 shows the proliferation of natural killer (NK) cells from human lymphocytes treated with various mushroom formulations of the present subject matter.

FIG. 10 shows the natural killing ability of NK cells when treated with various mushroom formulations of the present subject matter to induce death in leukemia cells.

FIG. 11 shows the natural killing ability of NK cells as percent activity when treated with various mushroom formulations of the present subject matter to induce death in leukemia cells.

FIG. 12 shows the ability of concentrations of a mushroom formulation of the present subject matter to inhibit oxidative damage within red blood cells (RBC) and that the components of the formulation are capable of crossing RBC membranes.

### DETAILED DESCRIPTION

### Definitions

As used herein, the terms “administer,” “administering,” and “administration,” refer to any method which, in sound medical practice, delivers the composition to a subject in such a manner as to provide a therapeutic effect.

The phrase “derivative” as used herein refers to any hydrate, solvate, salt, racemate, isomer, enantiomer, prodrug, metabolite, ester, or other analog or derivative of a particular chemical compound or molecule. The term “derivative” may also mean a modification to the disclosed compounds including, but not limited to, hydrolysis, reduction, or oxidation products, of the disclosed compounds. Hydrolysis, reduction, and oxidation reactions are known in the art.

The term “modulating” refers to the process of producing an effect on biological activity, function, health, or condition of an organism in which such biological activity, function, health, or condition is maintained, enhanced, diminished, or treated in a manner which is consistent with the general health and well-being of the organism.

As used herein, the phrases an “effective amount” or a “therapeutically effective amount” of an active agent or ingredient, or pharmaceutically active agent or ingredient, which are synonymous herein, refer to an amount of the pharmaceutically active agent sufficient enough to have a therapeutic effect upon administration. A therapeutically effective amount of the pharmaceutically active agent may, will, or is expected to cause a relief of symptoms. Effective amounts of the pharmaceutically active agent will vary with the particular condition or conditions being treated, the severity of the condition, the duration of the treatment, the specific components of the composition being used, and like factors.

The term “enhancing” the biological activity, function, health, or condition of an organism refers to the process of augmenting, fortifying, strengthening, or improving.

The term “eicosanoid” refers to any of the class of compounds derived from polyunsaturated fatty acids, such as arachidonic acid and linoleic acid, and involved in cellular activity. The term “lipoxygenase” refers to any of the class of enzymes that catalyze the incorporation of molecular oxygen into its lipid substrate.

As used herein, “subject” or “individual” or “animal” or “patient” or “mammal,” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay, prevention, or inhibition of the progression thereof. Treatment need not mean that the disease, disorder, or condition is totally cured. A useful composition herein needs only to reduce the severity of a disease, disorder, or condition, reduce the severity of symptoms associated therewith, provide improvement to a patient or subject's quality of life, or delay, prevent, or inhibit the onset of a disease, disorder, or condition.

As used herein, “wild crafted” refers to any mushroom species that is cultivated in a wild biological setting. All accessible portions of the mushroom may be incorporated into the mushroom formulation. The Chaga species (Inonotus obliquus) may be grown as sclerotia on birch trees and may then be called wild crafted. In one embodiment, the formulation of the present subject matter utilizes the wild crafted Chaga sclerotia from birch trees in addition to cultivated Chaga mycelia and fruit bodies, such as, for example, from Chaga grown on rice to generate a broad spectrum of the therapeutic compounds. The fruiting bodies of spores are easily collected from wild crafted Chaga sclerotia. However, the mycelium of wild crafted Chaga may be more difficult to harvest as compared to Chaga mycelium grown on rice.

Any concentration ranges, percentage range, or ratio range recited herein are to be understood to include concentrations, percentages or ratios of any integer within that range and fractions thereof, such as one tenth and one hundredth of an integer, unless otherwise indicated.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application. For example, “a” polymer refers to both one polymer or a mixture comprising two or more polymers.

Throughout the application, descriptions of various embodiments use “comprising” language; however, it will be understood by one of skill in the art, that in some specific instances, an embodiment can alternatively be described using the language “consisting essentially of” or “consisting of.”

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Other terms as used herein are meant to be defined by their well-known meanings in the art.

The Subject Compositions

The present subject matter relates to mushroom formulations useful for maintaining overall wellness and support of the immune system, and methods of making and using the same.

Mushrooms of the present subject matter may be gathered from the wild and/or cultivated. In one embodiment, cultivated mushrooms are grown on certified organic and biodynamic brown rice. In general, the formulation of the present subject matter comprises one or more mushroom components (from one or more species of mushroom) selected from the group consisting of mycelia, extracellular components in the mycelium biomass, fruiting bodies, and spores from the fruiting body. In one embodiment, the formulation comprises the fruiting bodies, spores, and mycelium of one or more mushrooms, and only the mycelium from one or more mushroom species.

In some embodiments, when mycelium is used in a formulation then extracellular components in the mycelium biomass may also present in the formulation. Extracellular components in the mycelium biomass may arise from sources selected from the group consisting of (a) components produced by the mushroom, (b) components produced by other organisms (non-limiting examples include microbes, plants, animalia, or other fungi) present in or near the mycelium biomass, (c) components naturally present in the mycelium biomass (non-limiting examples include minerals or vitamins naturally present in the soil in which the mycelium grows), and (d) components produced during the growth of the mushroom.

In one aspect, the subject matter involves a composition comprising a combination of mushrooms or components derived from mushrooms selected from the group consisting of Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Coriolus Tramentes versicolor, and Chaga Inonotus obliquus. In some embodiments, compositions of the present subject matter may also comprise other mushroom species not described herein.

Reishi species (for example, Ganoderma lucidum) is one mushroom that may be used in the formulation of the present subject matter. Reishi has been used as a medicine in China and Japan for over 4,000 years. In traditional herbal systems, this mushroom was used as a tonic for the five organs (lungs, liver, kidneys, heart, spleen), and was believed to increase longevity. Formulations of the present subject matter may contain the mycelium and fruiting bodies to capture a broad spectrum of the therapeutic compounds present in reishi. In one embodiment, a broad spectrum of compounds may be reliably obtained by using controlled cultivation methods, such as, for example, by growing on rice as well as additional fruiting bodies and spores that have been grown on wood pulp. The prominent role of reishi in traditional use is supported by hundreds of scientific studies that have been conducted in Asia as well as the United States and Europe examining reishi's therapeutic properties in in vitro, animal, and human studies.

Shiitake species (for example, Lentinula edodes) is another mushroom that may be in the formulation of the present subject matter. Shiitake mushrooms have been cultivated in China and Japan for a thousand years where they have been used as a tonic for the organ systems of the body. Modern scientific research has supported its abilities to be an adaptogen, immunosupportive agent, and for its cardiovascular supportive effects. In one embodiment, the formulation of the present subject matter contains both the mycelium and fruiting bodies of shiitake grown on rice to capture a broad spectrum of the therapeutic compounds present in Shiitake.

Lion's Mane species (for example, Hericium erinaceus) is another mushroom that may be in the formulation of the present subject matter. Lion's Mane has been used in traditional herbal systems for its ability to support the organ systems of the body, promote good digestion, general vigor, strength and nutrition. One embodiment of the present formula may contain both mycelia and fruiting bodies that have been grown on rice to capture a full spectrum of the therapeutic compounds present in the mushroom. Lion's Mane is useful as an antioxidant, for cognitive support, and for support of normal cell growth within the body.

Cordyceps species (for example, Cordyceps sinensis) is another mushroom that may be in the formulation of the present subject matter. Cordyceps is considered to be a moderately Yang tonic of the highest stature in traditional herbal systems. It is highly regarded in China as a tonic for those who are recovering from an illness or an operation, or after giving birth. In these cases, the Cordyceps helps the patient recover their physical power, to improve their appetite, and to protect the body from infection. Cordyceps is prized for its ability to enhance oxygen utilization by the body. Hundreds of studies have been conducted examining the wide variety of uses by cordyceps in a wide variety of countries.

Maitake species (for example, Grifola frondosa) is another mushroom that may be in the formulation of the present subject matter. Maitake is native to the northeastern part of Japan and North America, and is prized in traditional Chinese and Japanese herbology as a medicinal mushroom used to support the immune system. The D-fraction, a protein bound polysaccharide found in a hot water extract of maitake, is useful for its immune supportive and anti-cancer properties. In one embodiment, a formulation of the present subject matter uses the whole mycelium of maitake.

Poria species (for example, Poria cocos) is another mushroom that may be in the formulation of the present subject matter. Poria is one of the most widely used and respected herbs in Chinese herbalism after licorice. It is traditionally used in China as a tonic soup for the elderly and infirmed. Poria is useful for treating insomnia, restlessness, fatigue, sleep disorder, tension, and nervousness. Poria also has therapeutic benefits related to its anti-inflammatory actions.

Mesima species (for example, Phellinus linteus) is another mushroom that may be in the formulation of the present subject matter. Mesima has been used in herbal systems in Korea. Mesima is useful for immune support and for its anti-cancer actions.

Coriolus species (for example, Trametes versicolor) is another mushroom that may be in the formulation of the present subject matter. Coriolus has been a component of traditional Asian medicine for centuries and has been used to clear dampness, phlegm and heat. The two most well researched turkey tail products are PSP and PSK (Krestin), which are similar glycoproteins. The main chains of PSK and PSP are a 1,3β-D-glucan with polypeptide side chains. In one embodiment, a formulation of the present subject matter uses the whole mycelium of coriolus.

Chaga species (for example, Inonotus obliquus) is another mushroom that may be in the formulation of the present subject matter. Chaga has been used in Eastern European folk and botanical medicine since the 16th century. The sclerotia grown on birch trees have been shown to contain betulinic acid which has been demonstrated in modern research to possess anti-inflammatory activity. In one embodiment, the formulation of the present subject matter utilizes the wild crafted Chaga sclerotia from birch trees in addition to cultivated Chaga mycelia and fruit bodies, such as, for example, from Chaga grown on rice to generate a broad spectrum of the therapeutic compounds.

In one embodiment, the mycelium and a portion of the fruiting body stage of one or more of Reishi, Shiitake, Lion's Mane, and Chaga are included in the formula. In a further embodiment, the mycelium of one or more of Cordyceps, Maitake, Poria, Mesima, and Coriolus are included in the formula, but not their fruiting bodies or spores. In yet another embodiment, the mycelium and a portion of the fruiting body stage of Reishi, Shiitake, Lion's Mane, and Chaga are included in the formula along with the mycelium of Cordyceps, Maitake, Poria, Mesima, and Coriolus, but not their fruiting bodies or spores.

In another embodiment, each of the mushroom components may be present at any concentration. Such as, for example, at any concentration that will provide or promote a beneficial therapeutic effect. In one embodiment, the concentration of each species of mushroom as measured as a w/w % of total mushroom components may be selected from the group of ranges consisting of 0.1-5%, 0.2-5%, 1-10%, 5-15%, 5-25%, 5-95%, 10-90%, 10-50%, 10-30%, 15-30%, 20-40%, 20-60%, 30-80%, 30-70%, and 30-50%, wherein the total cumulative percentage of mushroom components is selected to be 100%.

In yet another embodiment, the formulation comprises the following mushroom components: by w/w % of the total mushroom components, 15-30% Reishi (Ganoderma lucidum) mycelium and fruiting bodies; 5-15% Shiitake (Lentinula edodes); 5-15% Lion's Mane (Hericium erinaceus); 5-15% Cordyceps (Cordyceps sinensis); 5-15% Maitake (Grifola frondosa); 5-15% Poria cocos (Poria cocos); 5-15% Mesima (Phellinus linteus); 5-15% Coriolus (Trametes versicolor); 3-15% Chaga (Inonotus obliquus); 3-15% Reishi Fruiting Bodies and Spores (Ganoderma lucidum); and 0.2-5% Chaga Sclerotia, Wild Crafted (Inonotus obliquus); and wherein the total sum percentage of the various mushroom components is 100%.

In one embodiment, the formulation comprises the following mushroom components: by w/w % of the total mushroom components, 15-30% Reishi (Ganoderma lucidum) fruiting bodies and mycelium; 5-15% Shiitake (Lentinula edodes) fruiting bodies and mycelium; 5-15% Lion's Mane (Hericium erinaceus) fruiting bodies and mycelium; 5-15% Cordyceps (Cordyceps sinensis) mycelium; 5-15% Maitake (Grifola frondosa) mycelium; 5-15% Poria cocos (Poria cocos mycelium); 5-15% Mesima (Phellinus linteus) mycelium; 5-15% Coriolus (Trametes versicolor) mycelium; 3-15% Chaga (Inonotus obliquus fruiting bodies and mycelium); 3-15% Reishi (Ganoderma lucidum) fruiting bodies and spores; and 0.2-5% Chaga Sclerotia, Wild Crafted (Inonotus obliquus); and wherein the total sum percentage of the various mushroom components is 100%.

In a further embodiment, the formulation comprises the following mushroom components: by w/w % of the total mushroom components, 18-25% Reishi (Ganoderma lucidum)) mycelium and fruiting bodies; 7-12% Shiitake (Lentinula edodes); 7-12% Lion's Mane (Hericium erinaceus); 7-12% Cordyceps (Cordyceps sinensis); 7-12% Maitake (Grifola frondosa); 7-12% Poria cocos (Poria cocos); 7-12% Mesima (Phellinus linteus); 7-12% Coriolus (Trametes versicolor); 7-12% Chaga (Inonotus obliquus); 3-8% Reishi fruiting bodies and spores (Ganoderma lucidum); and 0.5-3% Chaga Sclerotia, Wild Crafted (Inonotus obliquus); and wherein the total sum percentage of the various mushroom components is 100%.

In still another embodiment, the formulation comprises the following mushroom components: by w/w % of the total mushroom components, about 22.4% Reishi (Ganoderma lucidum); about 9.0% Shiitake (Lentinula edodes); about 9.0% Lion's Mane (Hericium erinaceus); about 9.0% Cordyceps (Cordyceps sinensis); about 9.0% Maitake (Grifola frondosa); about 9.0% Poria cocos (Poria cocos); about 9.0% Mesima (Phellinus linteus); about 9.0% Coriolus (Trametes versicolor); about 7.2% Chaga (Inonotus obliquus); about 5.6% Reishi Fruiting Bodies and Spores (Ganoderma lucidum); and about 1.8% Chaga Sclerotia, Wild Crafted (Inonotus obliquus).

In another embodiment, the formulation comprises the following mushroom components:

| | |
|---|---|
| Organic Reishi (Ganoderma lucidum)† | 224 mg |
| Organic Shiitake (Lentinula edodes)† | 90 mg |
| Organic Lion's Mane (Hericium erinaceus)† | 90 mg |
| Organic Cordyceps (Cordyceps sinensis)‡ | 90 mg |
| Organic Maitake (Grifola frondosa)‡ | 90 mg |
| Organic Poria cocos (Poria cocos)‡ | 90 mg |
| Organic Mesima (Phellinus linteus)‡ | 90 mg |
| Organic Coriolus (Trametes versicolor)‡ | 90 mg |
| Organic Chaga (Inonotus obliquus)† | 72 mg |
| Organic Reishi Fruiting Bodies and Spores | 56 mg |
| (Ganoderma lucidum) | |
| Chaga Sclerotia, Wild Crafted (Inonotus obliquus) | 18 mg |

(†Mycelium and fruiting bodies; ‡Mycelium)

In certain embodiments, the formulation comprises one or more components derived from two or more, three or more, four or more, five or more, six or more, seven or more, or eight or more mushroom species selected from the group consisting of Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Coriolis Tramentes versicolor, and Chaga Inonotus obliquus. In some embodiments, the formulation comprises one or more, two or more, three or more, four or more, five or more, six or more, seven or more, or eight or more components derived from the one or more of the mushroom species selected.

In one embodiment, the formulation comprises one or more components derived from two or more, three or more, four or more, five or more, six or more, seven or more, or eight or more of the mushroom species components selected from group consisting of Reishi Ganoderma lucidum fruiting bodies and mycelium, Reishi Ganoderma lucidum fruiting bodies and spores; Cordyceps sinensis mycelium, Maitake Grifola frondosa mycelium, Shiitake Lentinula edodes fruiting bodies and mycelium, Poria cocos mycelium, Lion's Mane Hericium erinaceus fruiting bodies and mycelium, Mesima Phellinus linteus mycelium, Turkey Tail Coriolis Tramentes versicolor mycelium, Chaga Inonotus obliquus fruiting bodies and mycelium; and wild crafted Chaga Inonotus obliquus.

Methods of the Present Subject Matter

The present subject matter relates to methods for modulating immune function, regulating the activity of lipoxygenases and cyclooxygenases, improving cardiovascular health, improving strength and endurance, and/or inhibiting cell proliferation diseases and disorders. In some embodiments, the present subject matter modulate pro-inflammatory and anti-inflammatory biochemical pathways in a manner that promotes beneficial health effects. In one embodiment, the composition provides a beneficial balancing of anti-inflammatory and pro-inflammatory function in animals, including in humans.

Therapeutically effective doses of the compositions of the present subject matter may be useful for preventing or treating inflammatory disorders, such as, for example, there are a wide range of conditions and diseases that are linked with chronic inflammation or that have an inflammatory component: These include, for example, acid reflux/heartburn, acne, allergies and sensitivities, Alzheimer's disease and other neurodegenerative diseases, appendicitis, autoimmune diseases such as lupus, asthma, atherosclerosis, arteriosclerosis, bronchitis, cancer, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, glomerulonephritis, heart disease, hepatitis, high blood pressure, insulin resistance, interstitial cystitis, joint pain/arthritis/rheumatoid arthritis/osteoarthritis, metabolic syndrome (syndrome X), myositis, myocarditis, nephritis, obesity, osteopenia, osteoporosis, pancreatitis, Parkinson's disease, pericarditis, periodontal disease, polyarteritis, polychondritis, prostatitis, psoriasis, scleroderma, sinusitis, Sjögren's syndrome, spastic colon, systemic candidiasis, tendonitis, urinary tract infection, vaginitis, and vasculitis.

Therapeutically effective doses of the compositions of the present subject matter may be useful for preventing or treating proliferative disorders. “Proliferative disease” means a class of diverse disorders and diseases characterized by a lack of control or poorly controlled cell division or proliferation. Proliferative diseases include disorders associated with an overgrowth of connective tissues, such as various fibrotic conditions, including scleroderma, atherosclerosis, rheumatoid arthritis, psoriasis, myeloproliferative diseases, idiopathic pulmonary fibrosis, scleroderma, juvenile arthritis, gouty arthritis, and liver cirrhosis, and conditions such as restenosis, arteriosclerosis, and proliferative diabetic retinopathy. Proliferative disorders also refers to neoplastic disorders including without limitation, anal cancer, bile duct cancer, colon cancer, esophageal cancer, gallbladder cancer, pancreatic cancer, small intestine cancer, stomach cancer, osteosarcoma, ovarian epithelial cancer, gestational trophoblastic tumor, uterine sarcoma, vaginal cancer, vulvar cancer, ovarian germ cell tumor, soft tissue sarcoma, hematopoietic malignancies including acute lymphoblastic leukemia, acute myeloid leukemia, and chronic myelogenous leukemia, lung cancer, small cell lung cancer, malignant mesothelioma, malignant thymoma, hypopharyngeal cancer, laryngeal cancer, nasopharyngeal cancer, oropharyngeal cancer, parathyroid cancer, salivary gland cancer, brain tumor, glioma, cerebellar astrocytoma, cerebral astrocytoma, ependymoma, medulloblastoma, adrenocortical carcinoma, pituitary tumor, islet cell carcinoma, bladder cancer, kidney cancer, penile cancer, Wilm's tumor, AIDS-related lymphoma, cutaneous T-cell lymphoma, Hodgkin's lymphoma, Ewing's sarcoma, skin cancer, hemangiomas of infancy and childhood, mycosis funoides, hairy cell leukemia, Kaposi's sarcoma, non-hodgkin's lymphoma, multiple myeloma, basal cell carcinoma, malignant melanoma, colorectal cancer, non-small cell lung carcinoma, bladder cancer, pancreatic carcinoma, renal cell carcinoma, neuroblastoma, bladder cancer, breast cancer, cervical cancer, liver cancer, sarcomas, thyroid cancer, endometrial cancer, uterine cancer, multiple myeloma, testicular cancer, retinoblastoma, colorectal cancer, oral cancer, rectal cancer, and prostate cancer.

The singular form “proliferative disease” includes any one or more diseases selected from the class of proliferative diseases, and includes any compound or complex disease state wherein a component of the disease state includes a disease selected from the class of proliferative diseases. The term also includes proliferative disorders refractory to treatment with other chemotherapeutics or that are refractory to treatment with other chemotherapeutics due to multidrug resistance. Proliferative disorders may also include those that involve excessive proliferation of cells and turnover of cellular matrix. The excessive cellular proliferation contributes significantly to the pathogenesis of several diseases, including cancer, atherosclerosis, rheumatoid arthritis, psoriasis, myeloproliferative diseases, idiopathic pulmonary fibrosis, scleroderma, and cirrhosis of the liver. Therapeutically effective doses of the compositions of the present subject matter may be useful for preventing or treating proliferative disorders

Therapeutically effective doses of the compositions of the present subject matter may be useful for preventing or treating cardiovascular diseases or disorders, such as, for example arteriosclerosis, atherosclerosis, vasculitis, myocarditis, congestive heart failure, and pericarditis. Therapeutically effective doses of the compositions of the present subject matter may be useful for preventing or treating other diseases or disorders, such as, for example, acid reflux/heartburn, acne, allergies and sensitivities, Alzheimer's disease and other neurodegenerative diseases, appendicitis, autoimmune diseases such as lupus, asthma, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, glomerulonephritis, hepatitis, high blood pressure, insulin resistance, interstitial cystitis, joint pain/arthritis/rheumatoid arthritis/osteoarthritis, metabolic syndrome (syndrome X), myositis, nephritis, obesity, osteopenia, osteoporosis, pancreatitis, Parkinson's disease, periodontal disease, polyarteritis, polychondritis, prostatitis, psoriasis, scleroderma, sinusitis, Sjögren's syndrome, spastic colon, systemic candidiasis, tendonitis, urinary tract infection, and vaginitis, The present composition is also expected to be of benefit in immunosuppressed humans which may occur through disease or treatment with, for example, steroid therapy.

In another embodiment, compositions of the present subject matter are effective for promoting eicosanoid synthesis and modulation beneficial for health.

In a further embodiment, the composition provides at least one therapeutic activity selected from the group consisting of (1) decreasing a pro-inflammatory or pro-proliferative biochemical response; (2) increasing an anti-inflammatory or anti-proliferative biochemical response; (3) decreasing the expression of genes associated with a pro-inflammatory or pro-proliferative biochemical response; (4) increasing the expression of genes associated with an anti-inflammatory or anti-proliferative biochemical response; (5) increasing the expression of TNF-α; (6) increasing the proliferation of NK cells; (7) increasing the natural killing ability of NK cells, and (8) increasing the migration of anti-inflammatory or anti-proliferative components to the site of an injury, disease, or disorder.

In another embodiment, the composition decreases the expression of at least one gene selected from the group consisting of (1) arachidonate 5-lipoxygenase; (2) histamine receptor H2; (3) interleukin 1 receptor, type I; (4) phospholipase A2, group X; (5) phospholipase A2, group IB; (6) phospholipase C, delta 1; (7) prostaglandin F receptor; and (8) tumor necrosis factor.

In yet another embodiment, the composition increases the expression of at least one gene selected from the group consisting of (1) adrenergic receptor, beta 1; (2) adrenergic receptor, beta 2; (3) annexin A3; (4) calcium channel, voltage-dependent beta 4 subunit; (5) cysteinyl leukotriene receptor 1; (6) hydroxyprostaglandin dehydrogenase 15 (NAD); (7) histamine receptor H1; (8) integrin alpha L; and (9) leukotriene A4 hydrolase.

Some other embodiments promote physical strength, endurance, and mental clarity.

The animal in all of the methods of the present subject matter disclosed herein may be a mammal such as a mouse, rat, cat, dog, horse, cow, or other domesticated animal, or a human. In a preferred embodiment, the animal is human. In addition to uses for treating human diseases, disorders, and conditions, the methods of the present subject matter may have veterinary applications.

Routes of Administration

In a certain embodiment, an orally administered composition is in the form of one or more capsules, one or more tablets, or one or more pills.

The subject compositions are preferably a dry composition comprising the dried mushroom components. In some embodiments, the dry composition is in the form of one or more capsules, one or more tablets, or one or more pills. In one embodiment, mushroom compositions are milled or ground before being prepared into a dosage form.

The subject compositions may also be delivered to the patient by means of a pharmaceutically acceptable carrier. Such carriers are well known in the art and generally will be in either solid or liquid form. Solid form herbal preparations which may be prepared according to the present subject matter include powders, tablets, dispersible granules, capsules, cachets and suppositories. In general, solid form preparations will comprise from about 5% to about 90% by weight of the active agent.

A solid carrier can be one or more substances which may also act as diluents, flavoring agents, solubilizers, lubricants, suspending agents, binders or tablet disintegrating agents; it can also be encapsulating material. In powders, the carrier is a finely divided solid which is in admixture with the viscous active compound. In tablets, the active compound is mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets prepared according to the present subject matter may be provided in chewable form, using techniques well known in the art.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby to solidify.

Liquid form preparations include solutions, suspensions, and emulsions. As an example may be mentioned water or water/propylene glycol solutions for parenteral injection. Liquid preparations can also be formulated in solution in aqueous polyethylene glycol solution. Aqueous solutions suitable for oral use can be prepared by dissolving the active component in water and adding suitable colorants, flavors, stabilizers and thickening agents as desired. Aqueous suspensions suitable for oral use can be made my dispersing the finely divided active component in water with a viscous material, i.e., natural or synthetic gums, resins, methylcellulose, sodium carboxymethylcellulose, and other well known suspending agents. Liquid pharmaceutical preparations may comprise up to 100% by weight of the subject active agent.

Solid form preparations which are intended to be converted, shortly before use, to liquid form preparations for either oral or parenteral administration are also contemplated as suitable carriers. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature (i.e., under refrigeration) in order to retard possible decomposition. The solid form preparations intended to be converted to liquid form may contain, in addition to the active material, flavorants, colorants, stabilizers, buffers, artificial and natural sweeteners, dispersants, thickeners, solubilizing agents, and the like. The liquid utilized for preparing useful liquid form preparations may be water, isotonic water, ethanol, glycerine, propylene glycol, and the like as well as mixtures thereof. Naturally, the liquid utilized will be chosen with regard to the route of administration. For example, liquid preparations containing large amounts of ethanol are not suitable for parenteral use.

The herbal preparations of the present subject matter may include one or more preservatives well known in the art, such as benzoic acid, sorbic acid, methylparaben, propylparaben and ethylenediaminetetraacetic acid (EDTA). Preservatives are generally present in amounts up to about 1% and preferably from about 0.05 to about 0.5% by weight of the pharmaceutical composition.

Useful buffers for purposes of the present subject matter include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame and the like and mixtures thereof may be utilized in amounts from about 0.001% to about 5% by weight of the composition.

Flavorants which may be employed in the herbal products of the present subject matter include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present subject matter include pigments which may be incorporated in amounts of up to about 6% by weight of the composition. A preferred pigment, titanium dioxide, may be incorporated in amounts up to about 1%. Also, the colorants may include other dyes suitable for food, drug and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857-884, which text is accordingly incorporated herein by reference.

Useful solubilizers include alcohol, propylene glycol, polyethylene glycol and the like and may be used to solubilize the flavors. Solubilizing agents are generally present in amounts up to about 10%; preferably from about 2% to about 5% by weight of the pharmaceutical composition.

Lubricating agents which may be used when desired in the instant compositions include silicone oils or fluids such as substituted and unsubstituted polysiloxanes, e.g., dimethyl polysiloxane, also known as dimethicone. Other well known lubricating agents may be employed.

The herbal preparation may also be prepared in a unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

It is not expected that compounds of the present subject matter will display significant adverse interactions with other synthetic or naturally occurring substances. Thus, a compound of the present subject matter may be administered in combination with other compounds and compositions useful, for example, for treating inflammation or cancer. In particular the compounds of the present subject matter may be administered in combination with other anti-inflammatory compositions or chemotherapeutic substances, and so forth.

The desired herbal formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the present subject matter.

Dosage

Dosage levels on the order of about 0.001 mg to about 100 mg per kilogram body weight of the active ingredient compounds or compositions are useful in the treatment of the targeted conditions, with preferred levels ranging from 200 mg per day to 1600 mg per day. The compounds and compositions of the present subject matter may usually be given in two or three doses daily. Starting with a low dose (200-300 mg) twice daily and slowly working up to higher doses if needed is a preferred strategy. The amount of active ingredient that may be combined with the carrier materials to produce a single dosage form will vary depending upon the host treated and the particular mode of administration.

It is understood, however, that a specific dose level for any particular patient will depend upon a variety of factors, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

### Example 1

One formulation of the present subject matter is termed “Life Shield Immunity” or “LSI”, and is as follows in Table I:

TABLE I

| Two capsule serving contains | Amt/serving |
|---|---|
| Organic Reishi (Ganoderma lucidum)† | 224 mg |
| Organic Shiitake (Lentinula edodes)† | 90 mg |
| Organic Lion's Mane (Hericium erinaceus)† | 90 mg |
| Organic Cordyceps (Cordyceps sinensis)‡ | 90 mg |
| Organic Maitake (Grifola frondosa)‡ | 90 mg |
| Organic Poria cocos (Poria cocos)‡ | 90 mg |
| Organic Mesima (Phellinus linteus)‡ | 90 mg |
| Organic Coriolus (Trametes versicolor)‡ | 90 mg |
| Organic Chaga (Inonotus obliquus)† | 72 mg |
| Organic Reishi Fruiting Bodies and Spores | 56 mg |
| (Ganoderma lucidum) | |
| Chaga Sclerotia, Wild Crafted (Inonotus obliquus) | 18 mg |

†Mycelium and Fruiting Bodies
‡Mycelium

All of the mushrooms were grown on certified organic & biodynamic brown rice. The finished formulation contains the mycelia, and the extracellular components produced during growth. The Reishi, Shiitake, Lion's Mane and Chaga were also grown to the fruiting body stage, so there were a portion of those fruiting bodies contained in the formula as well.

Cultures:

Reishi Ganoderma lucidum (full spectrum, 80-90% mycelium, 10-20% fruiting bodies)
Reishi Ganoderma lucidum (100% fruiting bodies)
Cordyceps sinensis (full spectrum)
Maitake Grifola frondosa (full spectrum)
Shiitake Lentinula edodes (full spectrum)
Poria cocos (full spectrum)
Lion's Mane Hericium erinaceus (full spectrum, 90-95% mycelium, 5-10% fruiting bodies)
Mesima Phellinus linteus (full spectrum)
Turkey Tail Tramentes versicolor (full spectrum)
Chaga Inonotus obliquus (full spectrum, 70-80% mycelium, 20-30% sclerotia)
Chaga Inonotus obliquus (wild crafted sclerotia)

Propagation and storage. Primary cultures were maintained at 4 deg C. on agar slants composed of a mixture of milled biodynamic organic brown rice, organic molasses, organic rice bran, agar and calcium carbonate. The organic brown rice was milled to (spec). Primary storage medium ingredients were mixed as follows: 30-60% Milled organic biodynamic brown rice; 2-5% Organic molasses; 5-15% Organic rice bran; 5-15% Agar; about 30-40 grams of the above dry mix was added to about 500-700 grams of water. The mixture was heated to dissolve in a flask, and dispensed into agar “slants”.

The Primary Culture Storage Medium Slants (PCSMS) are then prepared in screw cap 15×100 mm test tubes and autoclaved for 1-2 hr 121 deg C. under pressure (15-20 psi). These PCSMS were then stored at roughly 4 deg C. for up to 6 months. Primary cultures were inoculated under laminar flow onto these slants from frozen, lyophilized or PCSMS stock, grown for 5-15 days at 18-30 deg C. and then the PCSMS primary culture was stored at roughly 4 deg C.

Working Cultures (WC) were produced from the PCSMS primary culture by transferring a small portion of the surface of the PCSMS primary culture onto a petri dish previously prepared with the same nutrient base as was used in the PCSMS. This working culture was maintained at a temperature of 18-30 deg C. for 5-15 days. When confluence was 80-90%, the WC was transferred to roughly 4 deg C. for storage until needed.
Spawn Ingredients:

30-60% Biodynamic organic brown rice

2-5% Organic molasses

5-15% Organic rice bran

5-40 grams of above dry mix with 600 grams of water
Conditions: 60-75 deg F., sterile air sparged, constant agitation
Time: 3 days
Visual: Liquid spawn contains discrete spherules of suspended mycelium, medium showing signs of clearing.

Ingredients were mixed in cotton stoppered 2000 ml glass flasks and autoclaved 1-2 hours at 121 deg C. under at least 15 pounds pressure. Under sterile laminar flow, a small slice, roughly 2-4 cm² of Mushroom WC was added to the Starter flask and the flask was placed under continuous agitation in sterile laminar flow. Sterile air was introduced at a rate of about 10-20 L per hour through sparger in the bottom of the Starter flask for the duration of the starter growth cycle which may be 3-15 days.

Biodynamic organic brown rice or hardwood sawdust, calcium carbonate from, for example but not limited to algaes calcareas, lithothamnion, sea shells or similar may be used in the growth medium. 2-5 pounds of biodynamic organic brown rice or hardwood sawdust (preferably oak but also tulip poplar, or other hardwood) was placed in autoclavable filter patch bags. Calcium carbonate from, for example but not limited to, algas calcareas, lithothamnion sea shells, or similar may be added to the bags. The bags were then processed through an autoclave for 3-4 hours at 121 deg C. under at least 15 pounds pressure. 5-20 ml of Spawn was added to each bag under sterile laminar flow and the bags re then heat sealed and placed into a climate controlled growing room. (Temperature 18-28 deg C.) Culture timing depends on the species of mushroom and the extent of fruiting bodies required but may range from about 3-15 weeks.

Once the cultures have reached the full spectrum and or fruiting body stage, they were harvested and dried in a stainless chamber under flowing warm air (between 150-170 deg F.) until moisture level was between 2 and 8%. Mushroom cultures were then activated though steam treatment 1-2 hours at 120-125 deg C. under pressure of 15-20 psi. Dried mushrooms were milled through either a hammer or fitzmill to <50 mesh to <200 mesh, preferably to 90% through an 80 Mesh.

Another formulation of the present subject matter is described in Table II:

TABLE II

| Two capsule serving contains | Amt/serving |
|---|---|
| Organic Reishi (Ganoderma lucidum)† | 160 mg |
| Organic Lion's Mane (Hericium erinaceus)† | 500 mg |
| Organic Cordyceps (Cordyceps sinensis)‡ | 100 mg |
| Organic Poria cocos (Poria cocos)‡ | 100 mg |
| Organic Chaga (Inonotus obliquus)† | 80 mg |
| Organic Reishi Fruiting Bodies and Spores | 40 mg |
| (Ganoderma lucidum) | |
| Chaga Sclerotia, Wild Crafted (Inonotus obliquus) | 20 mg |

†Mycelium and Fruiting Bodies
‡Mycelium

### Example 2

### Anti-Inflammatory Activity of Compositions of the Present Subject Matter

Compositions of the present subject matter show anti-inflammatory activity evidenced by reduction of pro-inflammatory mediators and elevating the anti-inflammatory lipid mediators. The relative ability of compositions of the present subject matter to alter cyclooxygenase (COX) and lipoxygenase (LOX) metabolites as inflammatory mediators was measured.

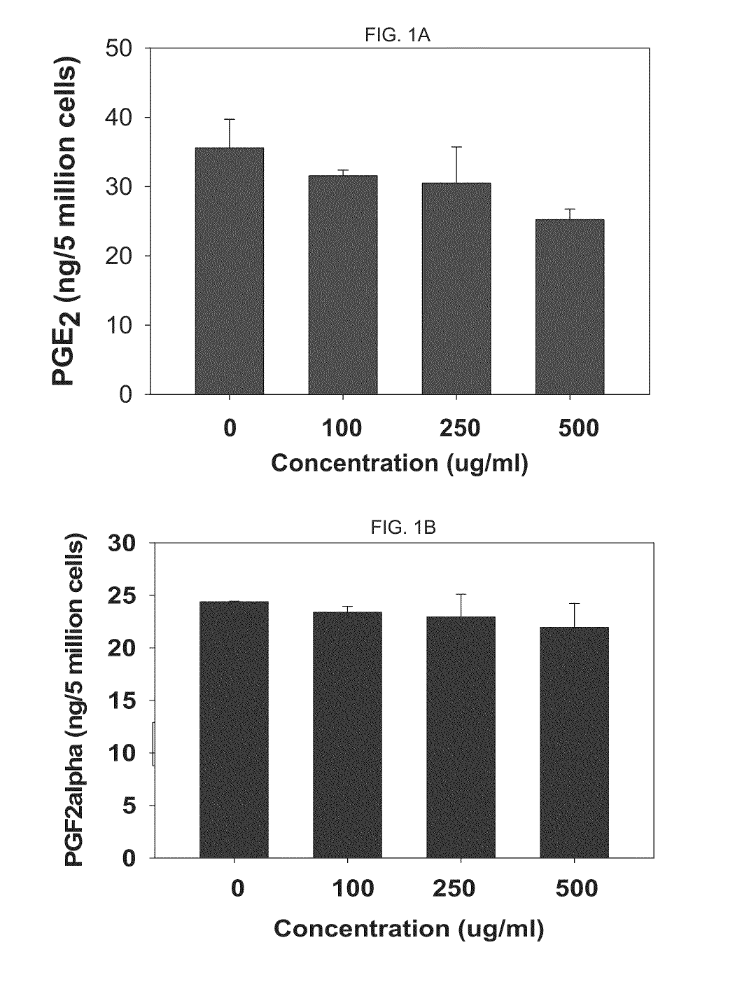

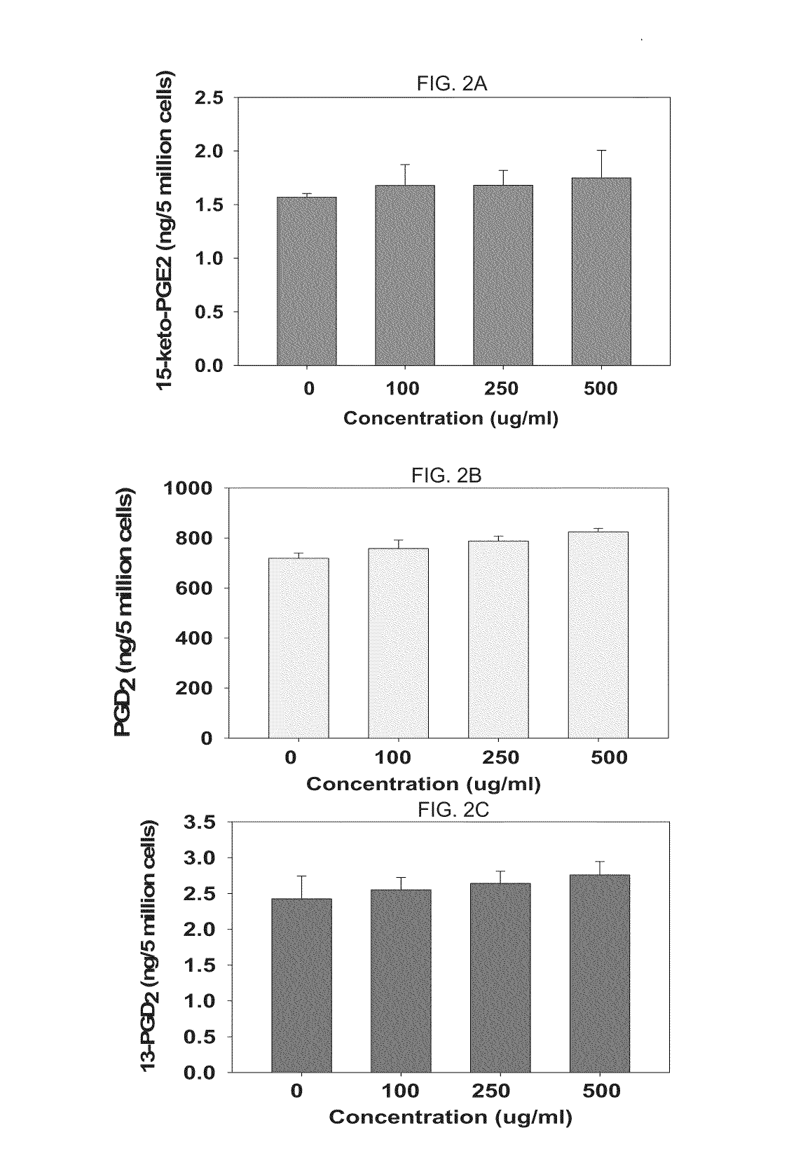

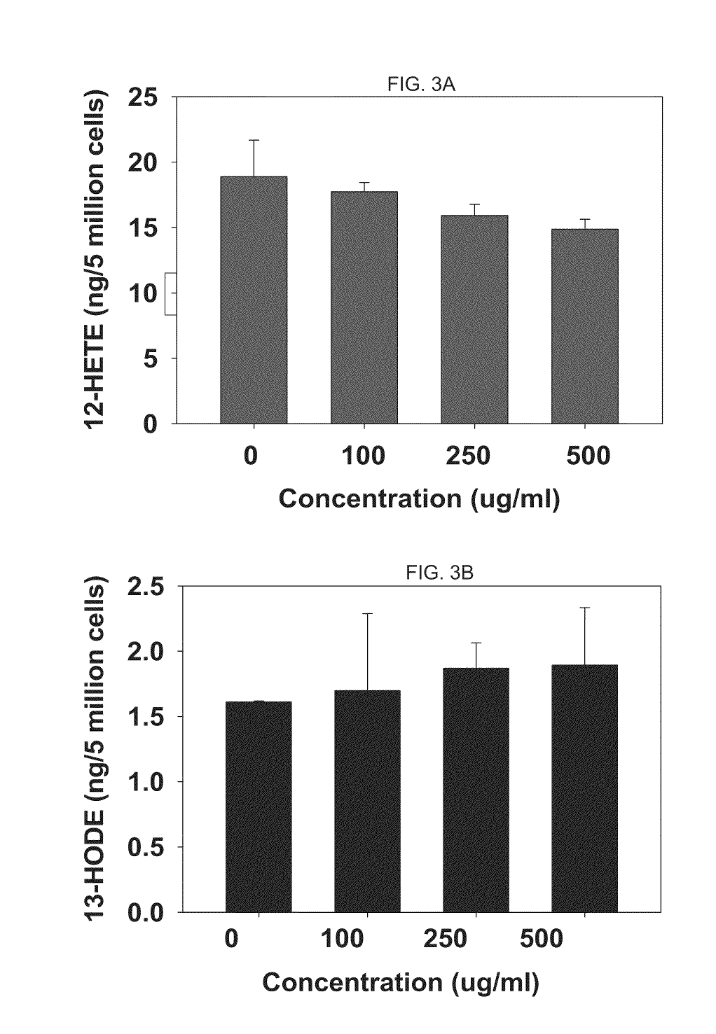

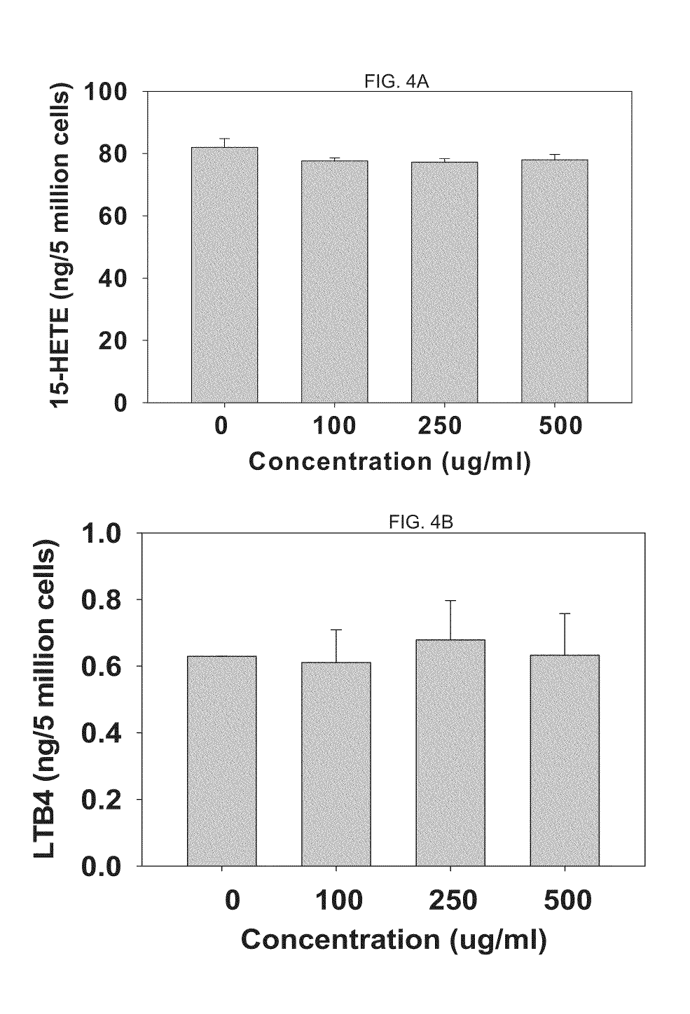

The relative effect LSI® on alteration of COX and LOX pathways indicated that LSI® (500 ug/ml) not only inhibited COX activity evidenced by significant reduction of PGE₂, and PGF₂ₐₗₚₕₐ formation (FIGS. 1A and 1B), but also increased the anti-inflammatory lipid mediators, such as 15-keto-PGE₂, PGD₂, 13,14-dihydro-15-keto-PGD2 (13-PGD₂) (FIGS. 2A, 2B, and 2C). Furthermore, this particular product also has ability to alter LOX pathways by reducing pro-inflammatory mediators, such as the 12-LOX product 12-HETE (37%) (FIG. 3A) or increasing the anti-inflammatory mediators, such as 15-LOX-1 product, 13-HODE (35%) (FIG. 3B). The levels of another 5-LOX metabolite, LTB4, and 15-LOX-2 metabolite, 15-HETE, exhibited minimal changes (FIGS. 4A and 4B). The effect of LSI on the COX and LOX pathways modulation of eicosanoids in Raw cells was concentration dependent. This data indicates compositions of the present subject matter were capable of altering both COX and LOX metabolic pathways and provide a beneficial balance between pro-inflammatory and anti-inflammatory pathways. These results support the anti-inflammatory activity of the compositions of the present subject matter.

FIGS. 1 and 2 show the effect of LSI on the formation of COX-2 derived metabolites in rat macrophage Raw264.7 cells. The assay was performed using 5 million intact A549 cells. Aliquots of LSI at concentrations indicated above were added to cell suspension and incubated for 10 min. The reaction was stopped by addition of 1N citric acid. Control was treated with similar amount of DMSO used in the preparation of LSI samples. Eicosanoids were then extracted using a hexane:ethyl acetate (1:1) solvent mixture; the extract was brought to dryness under nitrogen. Prostaglandins of interest formed during the incubation were extracted as previously described and then analyzed using our published LC/MS/MS method (Yang et al. Anal Biochem. 308: 168-177, 2002).The data indicate that the compositions of the present subject matter may have a strong effect on both inhibition of pro-inflammatory lipid mediators and increases of anti-inflammatory compounds through COX activity compared to that of cell treated with vehicle alone.

FIGS. 3 and 4 show the effect of LSI on formation of lipoxygenase products in rat macrophage Raw cells. The assay was performed using 5 million intact A549 cells. Similar experimental procedure was utilized in this study as previously described. The data indicate that the compositions of the present subject matter not only inhibit the pro-inflammatory lipid mediator 12-HETE, but also increases the anti-inflammatory metabolite 13-HODE in rat macrophage cells.

### Example 3

### The Effect of Compositions of the Present Subject Matter on the Inflammatory Gene Expression in Monocytes

To further study the role of compositions of the present subject matter in the inflammatory pathways, the inflammatory associated genes were studied in RAW monocytes treated with compositions of the present subject matter using the Inflammatory Microarray kit (Applied Biosystems). Expression of genes was analyzed by real time PCR instrument. Cells were plated on 100 mm plates and treated for 24 hrs with 250-500 ug/ml LSI in serum-free conditions. RNA was extracted from the cells using standard Trizol (Invitrogen) procedures. RNA was reverse transcribed following instructions in SuperScript® II First-Strand Synthesis Kit. The cDNA generated was prepared in Taqman Universal PCR Master Mix (Applied Biosystems). 100 ul was loaded into each well of the Inflammation Array Micro Fluidic Card (Applied Biosystems). It was centrifuged and then sealed. Software for the Inflammation Array was downloaded onto the 7900 HT Fast Real-Time PCR System (Applied Biosystems). The card was loaded and levels of DNA were measured.

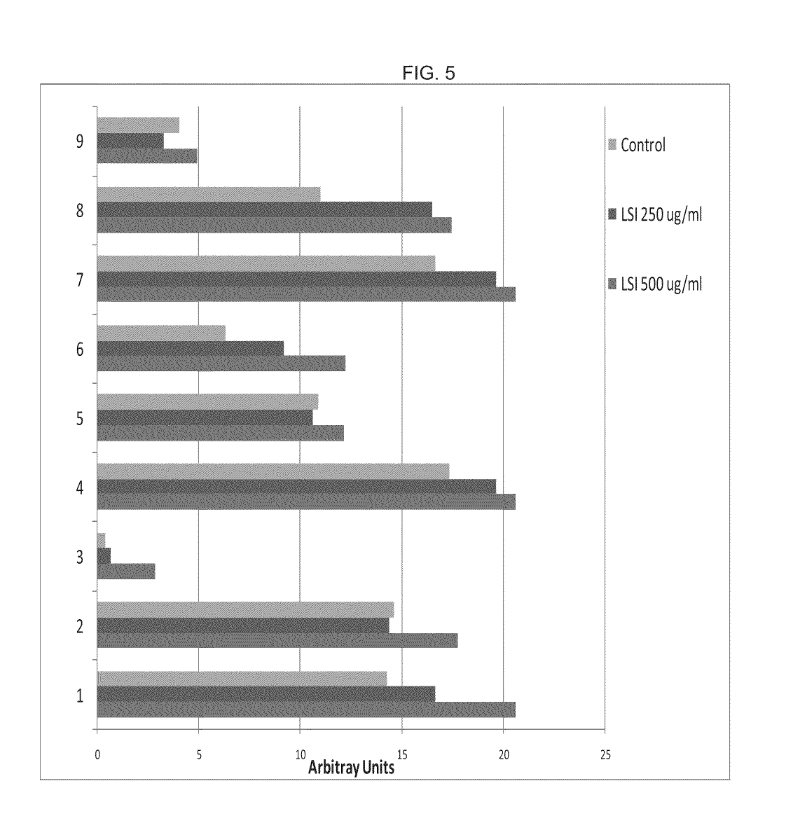

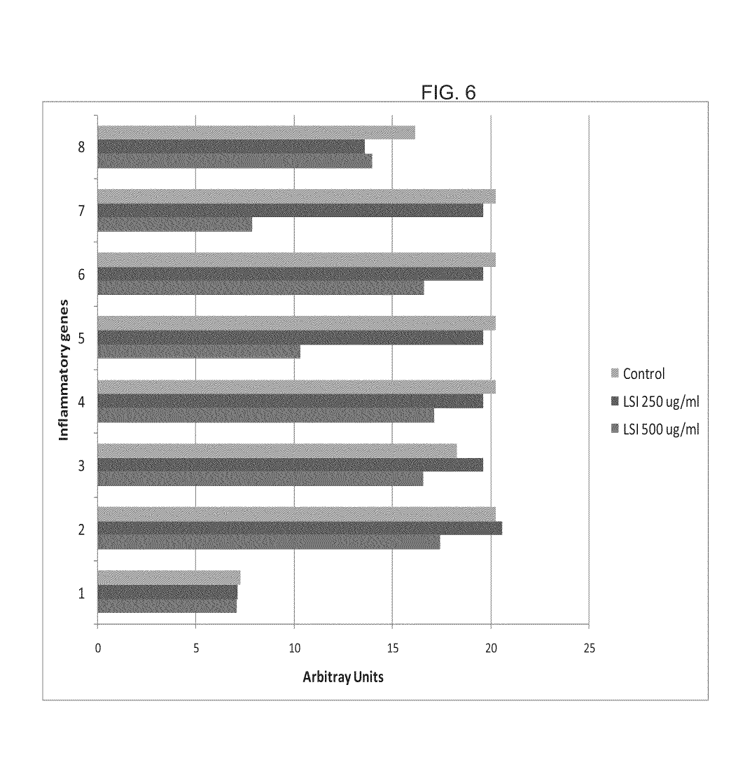

As shown in FIGS. 5 and 6, among a total of 95 genes examined, 17 genes were altered by the treatment of LSI and the alteration of these genes was in a concentration dependent manner. The formation of PGE2 was reduced, while its 15-PGDH metabolites, 15-keto was increased in RAW cells treated with a composition of the present subject matter (FIGS. 1 and 2). Interestingly, the expression of the gene associated with PGDH proteins was increased almost 100% in the cells treated with LSI (500 ug/ml) in comparison to that of cells treated with vehicle alone. The result indicates that the reduction of PGE₂ in RAW cells treated with LSI might not directly be due to the inhibition of synthetic enzyme of PGE₂, either COX-1 or COX-2, rather it was due to increased the degradation of this product by increasing the downstream metabolism of PGE₂. Given that the 15-PGDH has been reported to function as a tumor suppressor, the data indicate that compositions of the present subject matter have a great potential as a cancer preventive agent. Additionally, compositions of the present subject matter also markedly inhibited the PGF2alpha receptor and tumor necrosis factor in this assay.

FIGS. 5 and 6 show the expression of inflammatory associated genes in Raw cells treated with LSI. FIG. 5 demonstrates that LSI was capable of increased expression of enzymes such as 15-PGDH, which is tumor suppressor gene. In contrast, the reduced levels of inflammatory associated genes were shown in FIG. 6.

### Example 4

### The Effect of Compositions of the Present Subject Matter on Stimulating the Production of TNF-Alpha Production in Monocytes

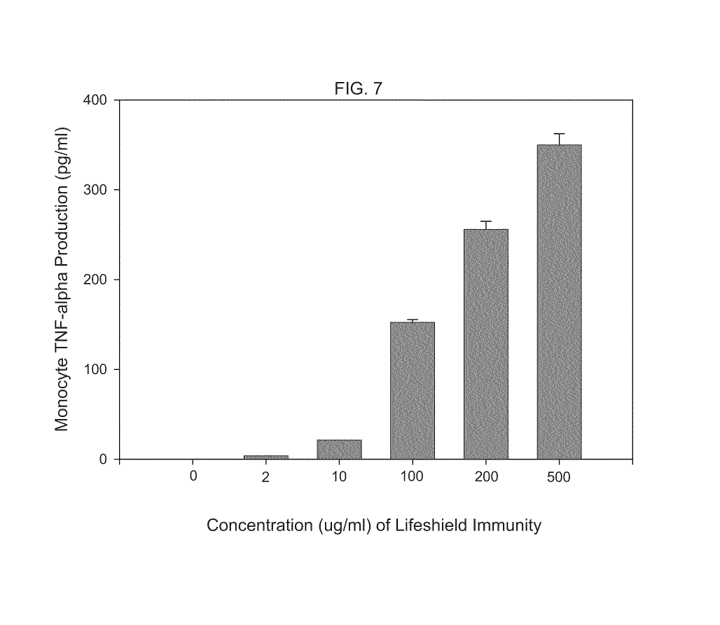

Polysaccharide extracts from plants can induce both pro- and anti-inflammatory cytokines, often altering their ratio in macrophages leading to immune stimulatory attributes. The induction and secretion of cytokines by compositions of the present subject matter was tested in monocyte model cell line Raw 2S4.7. Mouse monocyte cells (0.5×10⁶/ml) were starved overnight by growing in colorless DMEM containing 0.5% fetal bovine and antibiotics. On the following day, the plates were replaced with fresh medium and treated with varying doses of extracts (20-200 μg/ml for 24 h. The culture medium was collected and the TNFα produced and secreted into the medium by the cells was analyzed by ELISA procedure using the Quantakine kit from R&D systems, MN. The manufacturer's protocol was used for the assay. FIG. 7 shows the ability of the LSI composition to stimulate the production of TNF-alpha in monocytes which is an important and necessary step in an effective acute immune response.

Polysaccharide extracts from plants induce both pro- and anti-inflammatory cytokines, often altering their ratio in macrophages leading to immune stimulatory attributes. To test the induction and secretion of cytokines by New Chapter extracts, the mouse monocyte model cell line (Raw 264.7) was used. Mouse monocyte cells (0.5×10⁶/ml) were starved overnight by growing in colorless DMEM containing 0.5% fetal bovine and antibiotics. On the following day, the plates were replaced with fresh medium and treated with varying doses of extracts (20-200 ˜g/ml) for 24 h. The culture medium was collected and the TNF-α produced and secreted into the medium by the cells analyzed by ELISA procedure using the Quantakine kit from R&D systems, MN. The manufacturer's protocol was used for the assay.

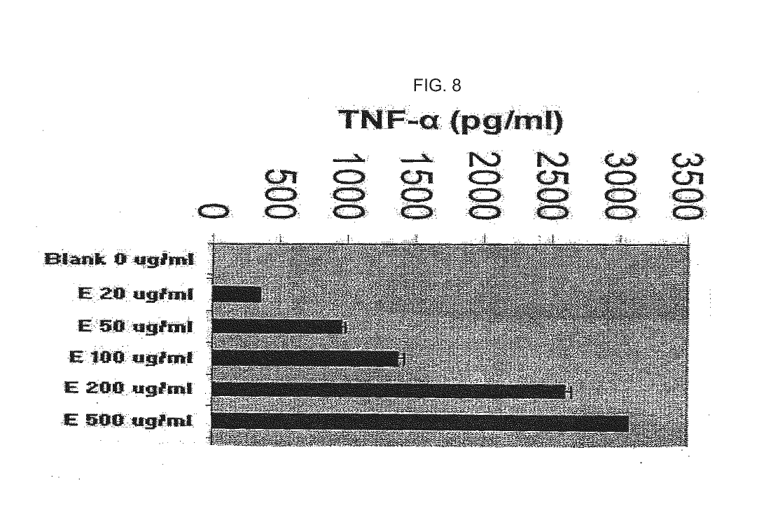

TNF-alpha (pg/ml) induced by various extracts are compared with a blank control (FIG. 8). LSI demonstrated a significant effect on TNF-α production compared to control.

### Example 5

### The Effect of Compositions of the Present Subject Matter on the Proliferation of NK Cells

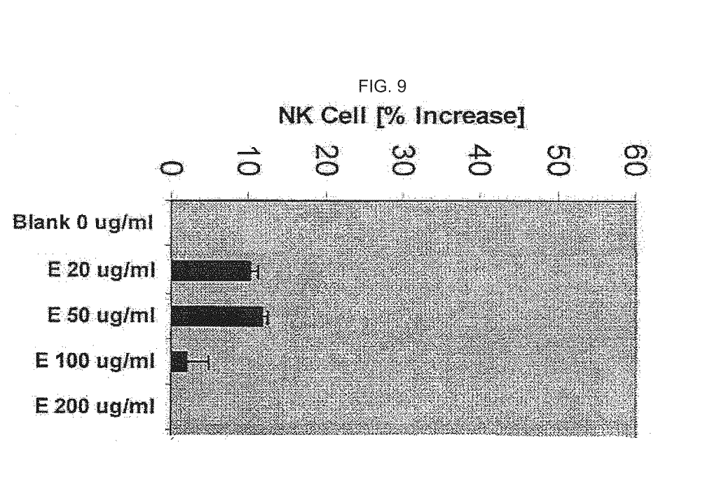

Immune stimulatory properties are associated with NK cell proliferation and function. NK cell proliferation was measured through the quantification of NK cells in normal lymphocytes from healthy volunteers upon exposure to each extract, Normal lymphocytes were isolated from healthy volunteers using histopaque 1077 gradient centrifugation. The cells were washed twice with PBS and resuspended in phenol-free RPMI medium supplemented with 10% fetal bovine serum and antibiotics (complete medium). Cells were plated in 12 well plates at 10⁶ cells/ml/well and treated with varying doses of extracts in a 5% CO₂ incubator at 37″C for 24 h. On the next day, lymphocytes were collected in 12×75 mm flow cytometry tubes and washed once with PBS. The cell pellet was stained with 20 IJI CD13/16+56 (FITC/PE), 10 μl of CD45 (APC), and 10 W. of CD19 (ECD) antibodies conjugated with different fluorochromes. The tubes were incubated for 30 minutes at room temperature. The cells were resuspended in 0.5 ml phenol-free complete RPMI medium and analyzed in a Coulter Elite flow cytometer using a 4-color flow cytometric protocol. LSI showed a significant effect on NK cell proliferation which appears to peak at around 50 μg/ml. (FIG. 9).

### Example 6

### The Effect of Compositions of the Present Subject Matter on the Natural Killing Ability of NK Cells

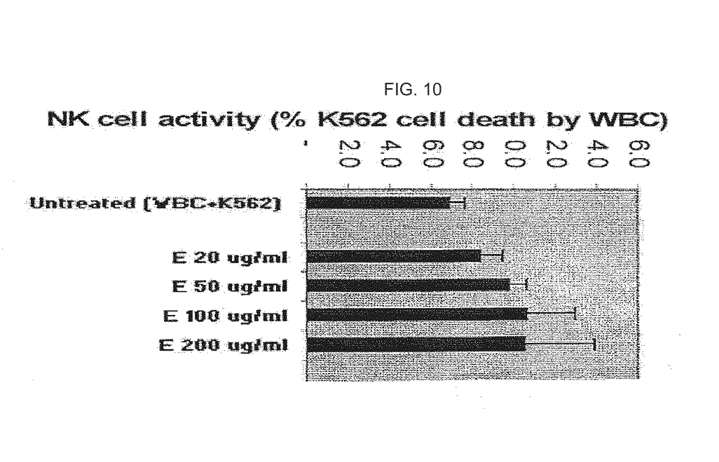

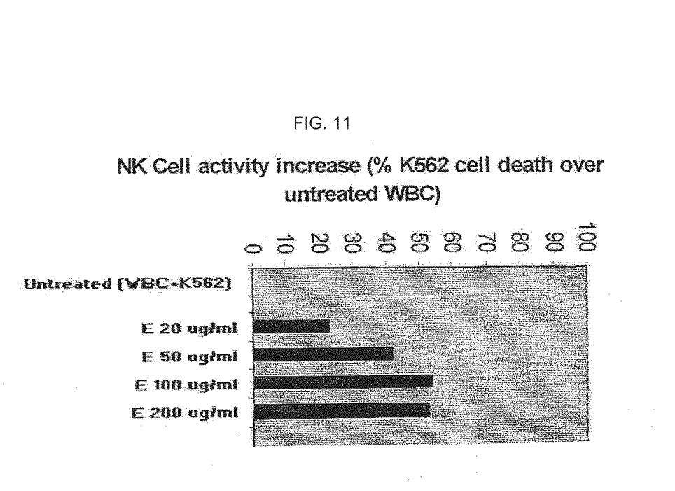

The natural killing ability of NK cells is considered an indicator of the immune stimulating ability of supplements and natural products. Therefore, the ability of NK cells in normal lymphocytes to induce death in leukemia cells was analyzed by co-incubating extract treated lymphocytes with K562 T-cell leukemia cells. Normal lymphocytes were isolated from healthy volunteers using histopaque 1077 gradient centrifugation. The cells were washed twice with PBS and resuspended in phenol-free RPMI medium supplemented with 10% fetal bovine serum and antibiotics (complete medium). Cells were plated in special 48-well deep plates (conical bottom) at 10⁶ cells/ml/well and treated with varying doses of extracts in a 5% CO. incubator at 3rC for 24 h. On the next day, log-phase K562 leukemic cells were labeled with PK1126 membrane dye (Sigma, Mo.) for 5 min according to manufacturer's protocol and 0.2×10⁶ cells each were added to the normal lymphocytes in the 48 well plates. The plate was centrifuged for 1 min at 400×g and returned to the CO₂ incubator for another 4 hours for inducing cell death. The cell mixture were stained with human specific FITC-labeled active form of caspase 3 antibody for 30 minutes at 4° C. by the procedure published earlier (Jerome et al. 2003; Nair et al. 2004). The stained cells were resuspended in 0.5 ml staining buffer and analyzed by a two color flow cytometry protocol with FL1 and FL2 measuring PKH26 and FITC, respectively. The percentage of K562 cells positive for PKH126 and FITC are dead cells induced by NK cells. The results of NK cell activity assay are shown in FIGS. 10 and 11. LSI showed the greatest enhancement of NK cell activity (relative to the untreated control).

### Example 7

### Modulating Migration of Anti-Inflammatory or Pro-Inflammatory Cells

Inflammation and immune response to perceived threats to the health of individuals are linked. That is, acute immune responses often involve recruitment of different populations of white blood cells which, in turn, are responsible for the release of pro-inflammatory mediators as well as signals for recruitment of even more specialized cell types. Polymorphonuclear cells (PMN) play a distinct role in immune surveillance as well as infiltration into sites of inflammation and this process is recognized as the first line of defense against bacterial infections.

PMN cells are migratory in nature. The migratory behavior is divided into at least two types: 1) random migration and 2) migration directed toward chemoattractant molecules. The ability of PMN to migrate towards specific chemoattractants such as interleukin-8 (IL-8) or leukotriene B4 (LTB4) have been well documented.

The effect of the present composition of mushroom products was tested on PMN cells to measure the effect of LSI on cell migration behavior. The data (not shown) indicated that LSI was able to induce effects on PMN migration at very low concentrations being effective at concentrations as low as 1 ng/ml.

The present subject matter being thus described, it will be understood that the same may be modified or varied in many ways. Such modifications and variations are not to be regarded as a departure from the spirit and scope of the present subject matter and all such modifications and variations are intended to be included within the scope of the following claims.

### Example 8

### Anti-Oxidant Activity of LIS in Human Cells

A cell based antioxidant protection assay (CAP-e) was used in human red blood cells (erythrocytes). The rationale behind the method is that this particular test allows assessment of antioxidant potential in a method that is comparable to the standard ORAC test, but only allows measurement of antioxidant materials that are able to cross the lipid bilayer of a human cell membrane.

Human RBCs were washed repeatedly in a physiological saline solution and then exposed to solutions of LIS. The product was prepared in PBS. During the incubation with LIS, any antioxidant components of LIS capable of crossing the RBC membrane would enter the cell. The RBCs were then washed to remove LIS components that were not absorbed by the cells and they were then loaded with a DCF-DA (Dichlorofluorescin diacetate) dye which becomes fluorescent upon exposure to reactive oxygen species. Oxidation was triggered by addition of the peroxyl free radical generator AAPH (2,2′-azobis-2-methyl-propanimidamide, dihydrochloride) and the fluorescence intensity evaluated. The low fluorescence intensity of untreated control cells served as a baseline, and RBC treated with AAPH alone served as a positive control for maximum oxidative damage. The observation of a reduced fluorescence intensity of RBC exposed to LIS and subsequently exposed to AAPH indicated that LIS contained antioxidants available to penetrate into the cells and protect them against oxidative damage.

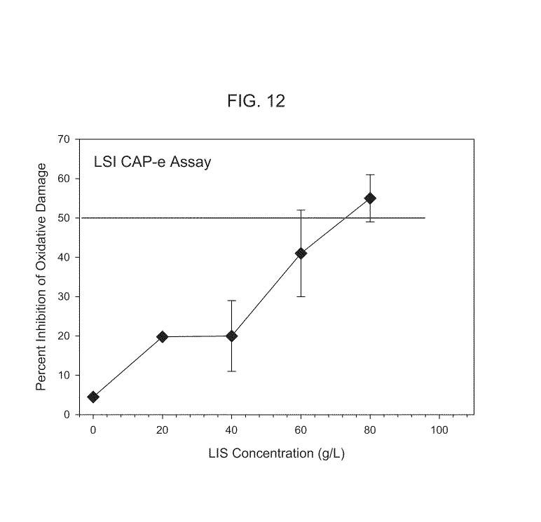

In the CAP-e assay, an IC50 of 46.7 g/L was reached for LSI prepared in PBS indicating that the product prepared in this manner contained compounds that were capable of crossing the cellular membrane of RBC and protecting the cells from oxidative damage. See FIG. 12. An 1050 value of 46.7 g/L translates to 0.68 CAP-e units per gram.

We claim:

1. A composition comprising one or more components derived from four or more of the mushroom species selected from the group consisting of Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Tramentes versicolor, and Chaga Inonotus obliquus.

2. The composition of claim 1, wherein the one or more components derived from the one or more of the mushroom species disclosed herein are derived from the mushroom mycelium biomass, mushroom fruiting bodies, mushroom spores, mushroom extracellular compounds in the mycelium biomass, or combinations thereof.

3. The composition of claim 1, wherein the one or more components are derived from five or more of the mushroom species selected from group consisting of Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Tramentes versicolor, and Chaga Inonotus obliquus.

4. The composition according to claim 1, comprising components derived from Reishi Ganoderma lucidum, Cordyceps sinensis, Poria cocos, Lion's Mane Hericium erinaceus, and Chaga Inonotus obliquus.

5. The composition according to claim 1, comprising components derived from Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey Tail Tramentes versicolor, and Chaga Inonotus obliquus.

6. The composition of claim 1, wherein the one or more components are derived from four or more of the mushroom species components selected from group consisting of Reishi Ganoderma lucidum fruiting bodies and mycelium, Cordyceps sinensis mycelium, Maitake Grifola frondosa mycelium, Shiitake Lentinula edodes fruiting bodies and mycelium, Poria cocos mycelium, Lion's Mane Hericium erinaceus fruiting bodies and mycelium, Mesima Phellinus linteus mycelium, Turkey Tail Coriolis Tramentes versicolor mycelium, and Chaga Inonotus obliquus fruiting bodies and mycelium.

7. The composition according to claim 1, comprising components derived from Reishi Ganoderma lucidum fruiting bodies and mycelium, Cordyceps sinensis mycelium, Maitake Grifola frondosa mycelium, Shiitake Lentinula edodes fruiting bodies and mycelium, Poria cocos mycelium, Lion's Mane Hericium erinaceus fruiting bodies and mycelium, Mesima Phellinus linteus mycelium, Turkey Tail Coriolis Tramentes versicolor mycelium, and Chaga Inonotus obliquus fruiting bodies and mycelium.

8. The composition according to claim 1, comprising components derived from Reishi Ganoderma lucidum fruiting bodies and mycelium, Cordyceps sinensis mycelium, Poria cocos mycelium, Lion's Mane Hericium erinaceus fruiting bodies and mycelium, and Chaga Inonotus obliquus fruiting bodies and mycelium.

9. A composition comprising, by w/w % of the total mushroom components, 15-30% Reishi (Ganoderma lucidum); 5-15% Shiitake (Lentinula edodes); 5-15% Lion's Mane (Hericium erinaceus); 5-15% Cordyceps (Cordyceps sinensis); 5-15% Maitake (Grifola frondosa); 5-15% Poria cocos (Poria cocos); 5-15% Mesima (Phellinus linteus); 5-15% Coriolus (Trametes versicolor); 3-15% Chaga (Inonotus obliquus); 3-15% Reishi Fruiting Bodies and Spores (Ganoderma lucidum); and 0.2-5% Chaga Sclerotia, Wild Crafted (Inonotus obliquus).

10. The composition of claim 9, comprising, by w/w % of the total mushroom components, 18-25% Reishi (Ganoderma lucidum); 7-12% Shiitake (Lentinula edodes); 7-12% Lion's Mane (Hericium erinaceus); 7-12% Cordyceps (Cordyceps sinensis); 7-12% Maitake (Grifola frondosa); 7-12% Poria cocos (Poria cocos); 7-12% Mesima (Phellinus linteus); 7-12% Coriolus (Trametes versicolor); 7-12% Chaga (Inonotus obliquus); 3-8% Reishi Fruiting Bodies and Spores (Ganoderma lucidum); and 0.5-3% Chaga Sclerotia, Wild Crafted (Inonotus obliquus).

11. The composition of claim 9, comprising, by w/w % of the total mushroom components, about 22.4% Reishi (Ganoderma lucidum); about 9.0% Shiitake (Lentinula edodes); about 9.0% Lion's Mane (Hericium erinaceus); about 9.0% Cordyceps (Cordyceps sinensis); about 9.0% Maitake (Grifola frondosa); about 9.0% Poria cocos (Poria cocos); about 9.0% Mesima (Phellinus linteus); about 9.0% Coriolus (Trametes versicolor); about 7.2% Chaga (Inonotus obliquus); about 5.6% Reishi Fruiting Bodies and Spores (Ganoderma lucidum); and about 1.8% Chaga Sclerotia, Wild Crafted (Inonotus obliquus).

12. A method of making a composition of claim 1, comprising
growing one or more of the selected mushrooms on certified organic and biodynamic brown rice;
harvesting at least one component from each selected species of mushroom grown; and
combining the components harvested from the various selected mushrooms into a dosage form.

13. A method of treating an inflammatory or proliferative disease or disorder, comprising administering a therapeutically effective dose of a composition of claim 1 to an animal in need thereof.

14. The method of claim 13, wherein the composition provides at least one therapeutic activity selected from the group consisting of (1) decreasing a pro-inflammatory or pro-proliferative biochemical response; (2) increasing an anti-inflammatory or anti-proliferative biochemical response; (3) decreasing the expression of genes associated with a pro-inflammatory or pro-proliferative biochemical response; (4) increasing the expression of genes associated with an anti-inflammatory or anti-proliferative biochemical response; (5) increasing the expression of TNF-α; (6) increasing the proliferation of NK cells; (7) increasing the natural killing ability of NK cells, and (8) increasing the migration of anti-inflammatory or anti-proliferative components to the site of an injury, disease, or disorder.

15. The method of claim 13, wherein the inflammatory disease or disorder is selected from the group consisting of, for example, acid reflux/heartburn, acne, allergies and sensitivities, Alzheimer's disease and other neurodegenerative diseases, appendicitis, autoimmune diseases such as lupus, asthma, atherosclerosis, arteriosclerosis, bronchitis, cancer, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, glomerulonephritis, heart disease, hepatitis, high blood pressure, insulin resistance, interstitial cystitis, joint pain/arthritis/rheumatoid arthritis/osteoarthritis, metabolic syndrome (syndrome X), myositis, myocarditis, nephritis, obesity, osteopenia, osteoporosis, pancreatitis, Parkinson's disease, pericarditis, periodontal disease, polyarteritis, polychondritis, prostatitis, psoriasis, scleroderma, sinusitis, Sjögren's syndrome, spastic colon, systemic candidiasis, tendonitis, urinary tract infection, vaginitis, and vasculitis.

16. The method of claim 13, wherein the proliferative disease or disorder is associated with an overgrowth of connective tissues selected from the group consisting of atherosclerosis, rheumatoid arthritis, psoriasis, myeloproliferative disease, idiopathic pulmonary fibrosis, scleroderma, juvenile arthritis, gouty arthritis, liver cirrhosis, restenosis, arteriosclerosis, and proliferative diabetic retinopathy.

17. The method of claim 14, wherein the composition decreases the expression of at least one gene selected from the group consisting of (1) arachidonate 5-lipoxygenase; (2) histamine receptor H2; (3) interleukin 1 receptor, type I; (4) phospholipase A2, group X; (5) phospholipase A2, group IB; (6) phospholipase C, delta 1; (7) prostaglandin F receptor; and (8) tumor necrosis factor.

18. The method of claim 14, wherein the composition increases the expression of at least one gene selected from the group consisting of (1) adrenergic receptor, beta 1; (2) adrenergic receptor, beta 2; (3) annexin A3; (4) calcium channel, voltage-dependent beta 4 subunit; (5) cysteinyl leukotriene receptor 1; (6) hydroxyprostaglandin dehydrogenase 15 (NAD); (7) histamine receptor H1; (8) integrin alpha L; and (9) leukotriene A4 hydrolase.
